(19) United States
(12) Patent Application Publication
(10) Pub. No.: US 2023/0083848 A1
(43) Pub. Date: Mar. 16, 2023

(54) RENAL VEIN ASPIRATION SYSTEM AND METHOD

(71) Applicant: Ali F. Aboufares, Englewood Cliffs, NJ (US)

(72) Inventor: Ali F. Aboufares, Englewood Cliffs, NJ (US)

(21) Appl. No.: 17/945,965

(22) Filed: Sep. 15, 2022

Related U.S. Application Data
(60) Provisional application No. 63/245,104, filed on Sep. 16, 2021.

(51) Int. Cl. A61M 60/33; A61M 60/104; A61M 60/20; A61M 60/515; A61M 60/851

(52) U.S. Cl. CPC A61M 60/33; A61M 60/104; A61M 60/20; A61M 60/515; A61M 60/851; A61M 2025/0031

(57) ABSTRACT

An aspiration system includes a plurality of catheters located at least partially within the lumen of an outer sheath. The outer sheath and the catheters are configured to slide relative to each other between a first state in which the distal ends of the catheters are located within the lumen of the outer sheath, and a second state in which the distal end and an end length section of each catheter is extended through the distal end of the outer sheath and outside of the lumen of the outer sheath. The end length section of each catheter is configured to spring or flare outward relative to the first state, when the outer sheath and the plurality of catheters are in the second state. The distal end of the outer sheath may be placed in an inferior vena cava or iliac vein in a transplant kidney patient, adjacent at least one renal vein, with the outer sheath and the plurality of catheters in the first state. Then, the system may be transitioned to the second state to cause the end length sections of the catheters to spring or flare outward adjacent the renal vein. The catheters are connected to a suction source for aspiration through the catheter end length sections.

### CROSS-REFERENCE TO RELATED APPLICATIONS AND CLAIM OF PRIORITY

This application claims the benefit of U.S. Provisional Application No. 63/245,104, filed Sep. 16, 2021, the content of which are fully incorporated herein by reference in its entirety.

### BACKGROUND

The present disclosure relates to medical aspiration systems and methods and, in particular examples, to systems and methods for renal vein aspiration.

Chronic kidney diseases and other renal diseases affect large populations, worldwide, with various degrees of severity. A hallmark of reduced renal function is an abnormally low glomerular filtration rate (GFR). This parameter (e.g., in ml./min.) can be measured and indirectly calculated using some variables obtained from the serum and urine of a patient. A normal GFR for an adult human is typically 60-100 ml/min, and a decrease may be associated with renal dysfunction. Typically, a lower GFR, indicates a more severe renal disease. For example, a GFR lower than 20 ml/min. could indicate end stage renal disease with the eventual need for dialysis to maintain survival.

Normally, the kidneys receive about one-fifth of the cardiac output. The volume of blood that enters the renal circulation per unit time is called the renal blood flow (RBF). The RBF and GFR are directly related; the higher the RBF, the higher the GFR.

RBF=(Renal artery pressure−Renal vein pressure)/Total renal vascular resistance.

Accordingly, to increase the RBF or renal forward flow and hence increase the renal perfusion, one can either elevate the Renal artery pressure, lower the Renal vein pressure or reduce the Total renal vascular resistance.

Much previous work has been devoted to improve the Renal artery pressure with medications and hemodynamic support devices. Other work has been performed for improving renal blood flow at the arterial side especially in low flow states as in cardiogenic shock etc. Similarly, work has been directed to lower the Total renal vascular resistance with medications. Also, improvements have been made in dialysis therapy with various filtration methodologies, such as hemo- and peritoneal dialysis.

In contrast to those efforts, certain examples of systems and methods described herein are configured to treat renal pathophysiology from the efferent or venous side of renal perfusion or filtration. It is believed that, by reducing the Renal vein pressure in a patient, one might be able to increase the RBF and hence the GFR of the patient. Particular examples described herein relate to aspirating renal venous blood with a suction device, to seek to lower the pressure in the renal vein and hence increase the differential pressure gradient across the kidneys. It is believed that this would increase the renal perfusion by increasing the RBF and consequently increase the GFR.

### BRIEF DESCRIPTION OF THE DRAWINGS

The above and other aspects and features of the present invention will become more apparent to those skilled in the art from the following detailed description of the example embodiments with reference to the accompanying drawings, in which:

FIG. 1 is a cross-section, side view along a longitudinal axis, showing an example of sections of a sheath and catheter components of a renal vein aspiration system, in a first state.

FIG. 2 is a cross-section, side view along a longitudinal axis, showing the example sheath and catheter component sections of FIG. 1, in a second state.

FIG. 3 is a side view of an end portion of one of the catheters in the system of FIG. 1.

FIG. 4 is a schematic view of a renal vein aspiration system including the sheath and catheter component in FIG. 1, in the first state.

FIG. 5 is a schematic view of a renal vein aspiration system including the sheath and catheter component in FIG. 1, in the second state.

FIG. 6 is a cross-section, side view along a longitudinal axis, showing a sheath of the system of FIG. 1, with a dilator and guide wire.

### DETAILED DESCRIPTION

Hereinafter, example embodiments will be described in more detail with reference to the accompanying drawings. The present invention, however, may be embodied in various different forms, and should not be construed as being limited to only the illustrated embodiments herein. Rather, these embodiments are provided as examples so that this disclosure will be thorough and complete, and will fully convey the aspects and features of the present invention to those skilled in the art. Accordingly, processes, elements, and techniques that are not necessary to those having ordinary skill in the art for a complete understanding of the aspects and features of the present invention may not be described. Unless otherwise noted, like reference numerals denote like elements throughout the attached drawings and the written description, and thus, descriptions thereof may not be repeated. Further, features or aspects within each example embodiment should typically be considered as available for other similar features or aspects in other example embodiments.

Certain terminology may be used in the following description for the purpose of reference only, and thus are not intended to be limiting. For example, terms such as “top”, “bottom”, “upper”, “lower”, “above”, and “below” could be used to refer to directions in the drawings to which reference is made. Terms such as “front”, “back”, “rear”, “side”, “outboard”, and “inboard” could be used to describe the orientation and/or location of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second”, and other such numerical terms referring to structures do not imply a sequence or order unless clearly indicated by the context.

It will be understood that when an element or feature is referred to as being “on,” “connected to,” or “coupled to” another element or layer, it can be directly on, connected to, or coupled to the other element or feature, or one or more intervening elements or features may be present. In addition, it will also be understood that when an element or features is referred to as being “between” two elements or features, it can be the only element or feature between the two elements or features, or one or more intervening elements or features may also be present.

The terminology used herein is for the purpose of describing particular embodiments and is not intended to be limiting of the present invention. As used herein, the singular forms “a” and “an” are intended to include the plural forms as well, unless the context clearly indicates otherwise. It will be further understood that the terms “comprises,” “comprising,” “includes,” and “including,” “has, ” “have, ” and “having,” when used in this specification, specify the presence of the stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list.

As used herein, the term “substantially,” “about,” and similar terms are used as terms of approximation and not as terms of degree, and are intended to account for the inherent variations in measured or calculated values that would be recognized by those of ordinary skill in the art. Further, the use of “may” when describing embodiments of the present invention refers to “one or more embodiments of the present invention.” As used herein, the terms “use,” “using,” and “used” may be considered synonymous with the terms “utilize,” “utilizing,” and “utilized,” respectively.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

Example embodiments relate to aspiration systems for use in medical environments and applications, and methods of making and using the same. Particular examples relate to systems and methods for renal vein aspiration. In certain examples, such systems and methods further include a venous return.

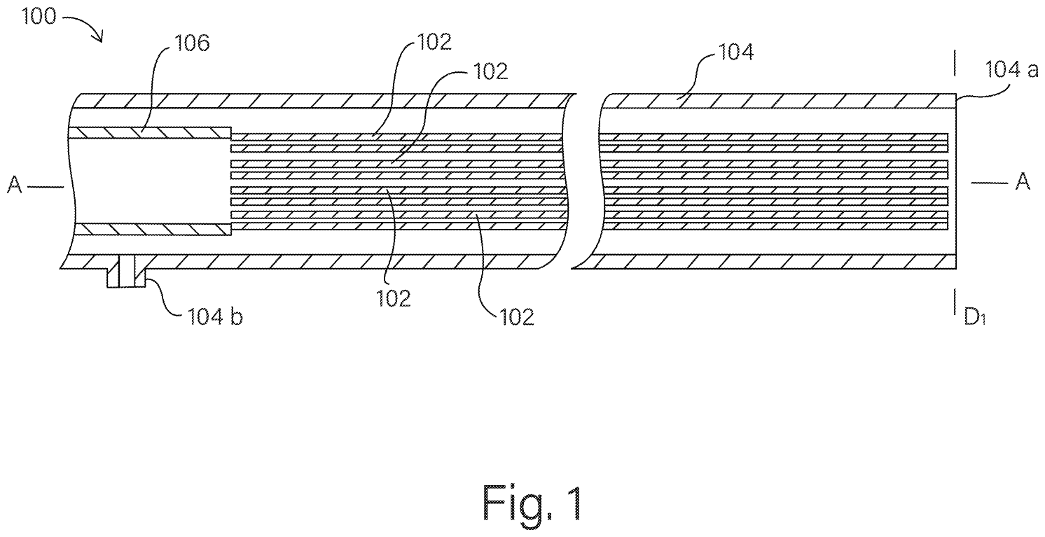

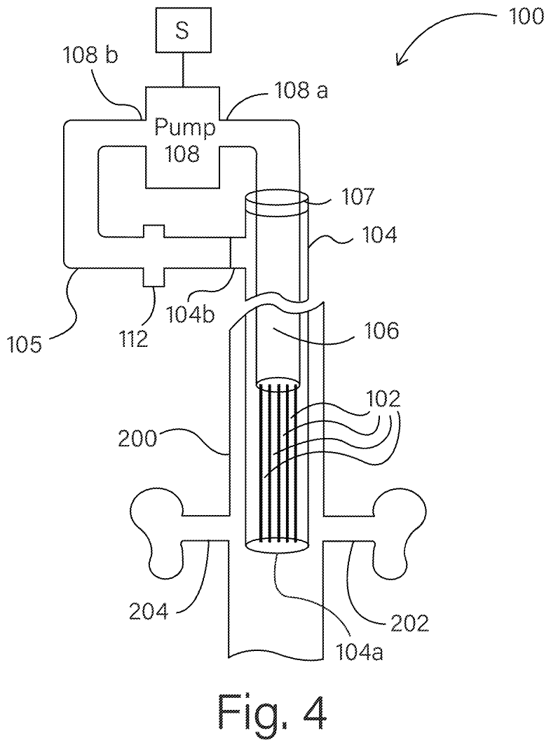

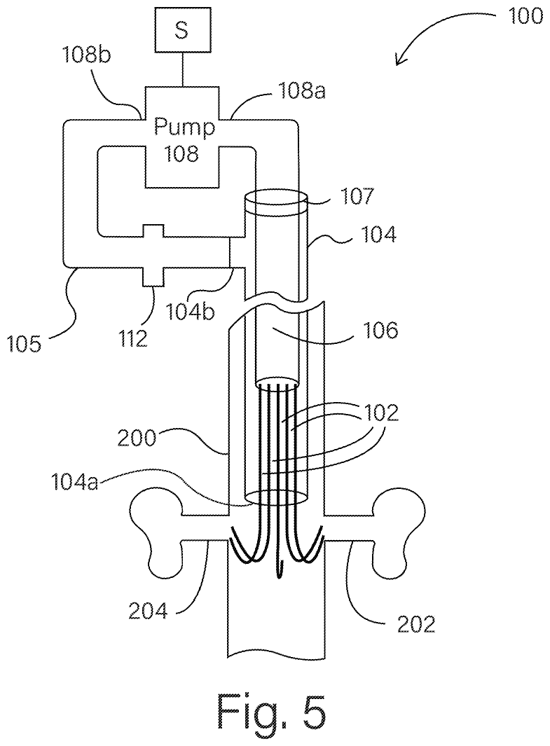

Examples of certain components of a renal vein aspiration system 100 are shown in FIGS. 1-3. The diagrams in FIGS. 4 and 5 represent an example of the system 100 (in a placement position and in an operating position, respectively), within a portion of a patient's inferior vena cava 200, at the level of the renal veins 202, 204. Further examples relate to methods of implanting catheter components of the system 100 in a patient, connecting those components to a pump component (or other suction source), and operating the system to provide renal vein aspiration treatment. Yet further examples relate to methods of making the system 100 and components thereof.

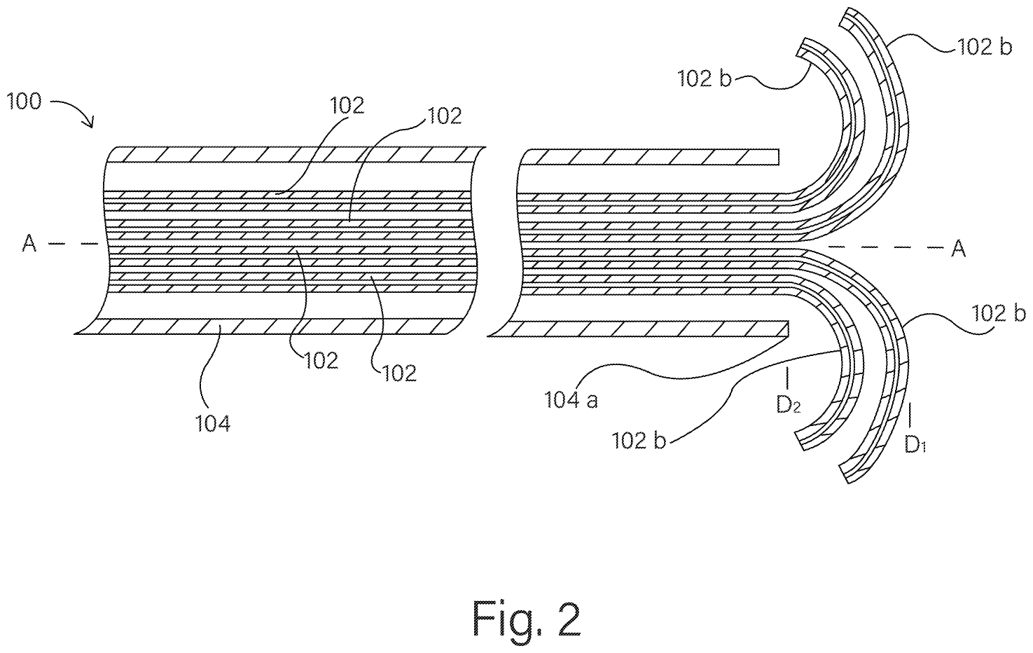

The renal vein aspiration system 100 includes a plurality of catheters 102 located within an inner lumen of an outer sheath 104. The sheath 104 has a longitudinal dimension (represented by the axis A in FIGS. 1 and 2) and may be flexible along its longitudinal dimension, so that it can bend and be guided through one or more venous lumens in a patient. Each of the catheters 102 has a longitudinal dimension arranged along the longitudinal dimension of the sheath 104. The system 100 may include any suitable number of catheters 102 within the outer sheath 104, depending upon the context of use, the size of a patient, or other criteria. In certain examples, the system 100 includes between four and ten catheters 102 within the outer sheath 104. In other examples, the system may include a number of catheters 102 outside of that range.

In FIGS. 1-3, the catheters 102 and the sheath 104 are shown with curved-line breaks in the center or at one end of their length dimension (the horizontal dimension in FIGS. 1-3), to indicate that the lengths of those components may be any suitable length for a desired application of use. The configurations of the components in the various length dimensions represented by those breaks may be the same as or consistent with the configurations on each side of the breaks.

The catheters 102 and sheath 104 are slidable relative to each other, such that the sheath 104 may be selectively slid off of distal end portions of the plurality of catheters 102. More specifically, the system 100 is configured such that the distal ends (the ends on the right side of FIG. 1) of each of the catheters 102 may be arranged within the inner lumen of the sheath 104 or may be approximately coplanar with a distal end of the sheath 104 and may be covered by the sheath. In that position, the system 100 is in a first state or a placement state. In the first state, the distal end of the sheath 104 (or of the catheters 102) may be located at a first inserted distance D1 within a placement site as shown in FIG. 1. In certain examples described herein, the placement site may be within the lumen of an inferior vena cava nt or iliac vein system (such as, but not limited to an iliac vein of a kidney transplant patient), or a similar venous or arterial lumen of a patient.

The system 100 is configured such that, from the first state, the distal end of the sheath 104 may be selectively slid or retracted to a second inserted distance D2 (e.g., a suitable distance off of the distal ends of the catheters 102), as shown in FIG. 2. In that position, the system 100 is in a second state or a treatment state. In the second or treatment state, a length portion of the distal end of each of the catheters 102 is external to and not covered by the sheath 104, as shown in FIG. 2.

In certain examples as described herein, each of the catheters 102 may be flexible along its length dimension, and may be made of a material or configuration (or both) that causes the uncovered length portion of the distal end of the catheters 102 to spring or flare outward relative to the axial dimension A of the sheath 104, in response to the sheath 104 being slid or retracted to its second state. More specifically, when the catheters 102 and the sheath 104 are in the first state (as shown in FIG. 1), the distal ends of the catheters 102 may be held within and abutted against the sheath 104, and retained from springing or flaring outward. However, when the catheters 102 and the sheath 104 are in the second state (as shown in FIG. 2), the sheath 104 no longer abuts and retains the uncovered length portions of the distal ends of the catheters and allows those end portions to spring or flare outward relative to the axis A. In particular examples, the distal end portions of the catheters 102 are configured to spring or flare outward with variable degrees of flexion angles depending on the surrounding anatomy. In certain examples, the end portions of those catheters 102 may be configured to become juxta-positioned to the renal veins ostia of a patient, when sprung or flared outward.

Each catheter 102 includes a hollow tube or tubing having an inner lumen through which fluid may flow. Each end of the catheter tubing may be open to the inner lumen. In certain examples, each catheter 102 has a relatively small outer diameter or outer dimension (OD). In particular examples, one or more (or each) catheter 102 is a micro-catheter and has an OD in the range of about one millimeter to about three millimeters. In other examples, one or more (or each) catheter 102 may have an OD outside of that range.

In particular examples, each catheter 102 is a single lumen catheter. A single lumen catheter configuration can simplify manufacturing and reduce costs. In addition, a single lumen catheter configuration can allow each catheter 102 to have a maximum inner diameter (ID) for a given catheter outer diameter (OD), for increased flow rate. In other examples, one or more (or each) catheter 102 may have an ID outside of that range. Further, in other examples, one or more (or each) of the catheters 102 may have a tubing with more than one lumen.

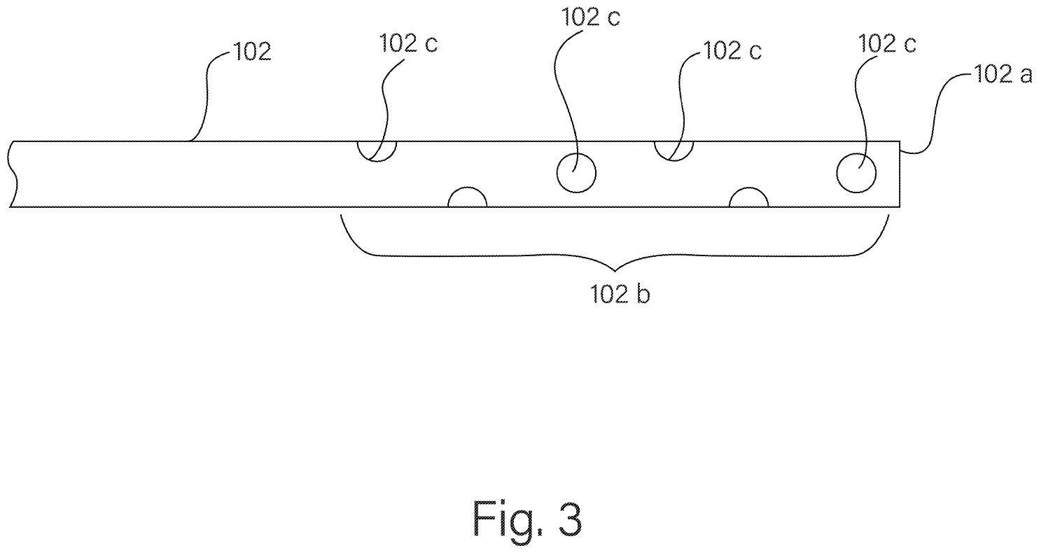

Each catheter 102 has a distal end (shown on the right side of FIGS. 1-3, and labeled 102a in FIG. 3). In certain examples, each catheter 102 has a length portion (labeled 102b in FIG. 3) extending from the distal end 102a toward the catheter's opposite, proximal end (not shown in FIG. 3) a defined length. In particular examples, the defined length of the length portion 102b is about equal to and corresponds to the length of the distal end portion that springs or flares outward as shown in FIG. 2. In other examples, the defined length of the length portion 102b is longer than, or is shorter than the length of the distal end portion that springs or flares outward as shown in FIG. 2. The length portion 102b may have any suitable length depending upon the application of use of the system and, in certain examples, is within a range of about one inch to about three inches (or is about two inches).

As shown in FIG. 3, the catheter wall along the length portion 102b of one or more (or each) of the catheters 102 may be provided with one or more (or a plurality of) openings 102c into the fluid flow lumen of the catheter. In certain examples, a plurality of openings 102c are provided at different positions along the axis A or around the axis A (or along and around the axis A). The openings 102c are configured to increase the flow rate of fluid into (or out of) the distal end portion of the catheters 102, as described herein. Alternatively or in addition, the plurality of openings 102c are configured to provide multiple flow passages to allow continued fluid flow into (or from) the catheter 102, in the event that one or more of the openings or the distal end opening becomes obstructed by a venous wall. In particular examples, the openings 102c are provided on the length portion 102b, but not on the rest of the axial length of the catheter 102.

The axial length of each catheter 102 (from the distal end 102a to the catheter's opposite, proximal end (not shown in FIG. 3)) may be any suitable length selected for a particular application of use. In certain examples, the axial length of each catheter 102 is in the range of about 40 centimeters to about 150 centimeters. In other examples, the axial length of each catheter 102 may be outside of that range. Each catheter 102 may have a circular cross-section shape (taken perpendicular to the axis A). In other examples, the catheter tubing may have a cross-section shape that is not a circle, such as, but not limited to an oval, another curved shape, a polygon or a shape having a combination of curved and straight edges. Similarly, the cross-section shape of the lumen of each catheter 102 may have a circular or another shape, and may correspond to (be the same shape as) the outer cross-section shape of the catheter tubing. In other examples, the cross-section shape of the catheter lumen may be a different shape relative to the outer cross-section shape of the catheter tubing.

The tubing of each catheter 102 may be made of a material that is compatible with blood or other fluids intended to be conveyed through the tubing, and with other materials to which the tubing may come into contact or be connected, in the intended environment of use. In certain examples, the catheter tubing is made of a material that is biologically compatible, for use in contexts in which the tubing is in contact or connected with a biological entity (such as a human patient or another biological entity). In certain examples, the catheter tubing is treated in one or more processes for enhancing biologically compatible such as, but not limited to cleaning, sterilizing, coating with Heparin, or the like.

In certain examples the tubing of each catheter 102 is made of a material that is suitable for medical uses, including but not limited to materials compatible with, and suitable for, implanting or partially implanting into a patient or other biological entity. Alternatively or in addition, the catheter tubing material is selected to provide end portions with a desired spring memory and sufficient rigidity and resilience to spring or flare outward, as shown and described with regard to FIG. 2. In particular examples, the material is also selected to provide sufficient flexibility and compliance to flex with the sheath 104, and to allow the end length portions 102b to spring or flare outward and contact a venous wall with no, or a reduced likelihood of, damage to the venous wall. Such materials may include, but are not limited to a plastic material, a thermoplastic elastomer material, a silicon material, a polymer material, a synthetic rubber material, Nitinol, stainless steel, or the like. However, for other contexts and applications of use, the catheter tubing may be made of other materials suitable and compatible with those contexts and applications. In certain examples, the tubing material for one or more (or each) of the catheters is pre-shaped by molding, heat treating, or other techniques have an end length portion 102b defining an outward-directed flare, one or more curls or bends, a helical configuration, a spiral configuration, or a combination thereof.

The tubing of each catheter 102 may be made by any suitable manufacturing process including, but not limited to extrusion, molding, machining, shrinking over a mandrel, combinations thereof, or the like. The spring or flared end portion of the tubing of each catheter 102 may be formed by any suitable process or mechanism including, but not limited to a curved or flared shape provided by molding, bending, machining, extruding or the like, or by a natural spring force of the tubing material (or a combination thereof). In other examples, the spring or flared end portion of the tubing of each catheter 102 may be formed by a shaped spring metal coil or other spring structure imbedded in the tubing wall of the catheter, or by other suitable process or mechanism.

The outer sheath 104 includes a hollow tube or tubing having at least one inner lumen through which the catheters 102 extend. The sheath 104 has a distal end 104a (on the right side of FIGS. 1 and 2) that is open to the inner lumen(s). The sheath 104 has an outer diameter or outer dimension (OD) that is sufficiently small to fit within an environment of use, such as, but not limited to a section of an inferior vena cava of a patient as described herein.

The lumen (or lumens) of the sheath 104 has an inner diameter or inner dimension (ID) that is sufficiently large to receive all of the catheters 102 along its axial length dimension (or all of the catheters 102 within one or more outflow catheters 106 described below). In particular examples, the sheath 104 has a single lumen. In other examples, the sheath 104 may have more than one lumen through which the catheters 102 extend.

In particular examples, the sheath 104 includes a venous return port 104b that is configured to connect a distal end of a return fluid tube 105 in fluid flow communication with the inner lumen of the sheath 104, as described below. The venous return port 104b may be located at a suitable distance from the distal end 104a of the sheath 104, such that any increase in blood pressure at the return port 104b does not affect (or significantly affect) the blood pressure at the distal end 104a of the sheath. The venous return port 104b may include any suitable releasable connection mechanism for releasably connecting a return tube in fluid flow communication such as, but not limited to a Luer connector or other medical quality fluid connector. In other examples, the return tube 105 may be connected in fluid flow communication with the port 104b through a permanent or non-releasable connection. The return tube 105 may be any tubing suitable for conveying blood or other fluid as described herein, including, but not limited to medical grade, flexible tubing.

In certain examples the sheath 104 is made of a material that is suitable for medical uses, including but not limited to materials compatible with, and suitable for, implanting or partially implanting into a patient or other biological entity. In particular examples, the sheath material is also selected to provide sufficient combination of rigidity, flexibility and compliance to allow the sheath 104 to be inserted into and fed through a venous system of a patient, with no or a reduced likelihood of, damage to the venous wall. Such materials may include, but are not limited to a plastic material, a thermoplastic elastomer material, a silicon material, a polymer material, a synthetic rubber material, Nitinol, stainless steel, or the like. However, for other contexts and applications of use, the sheath 104 may be made of other materials suitable and compatible with those contexts and applications. The sheath 104 may be made by any suitable manufacturing process including, but not limited to braiding, extrusion, molding, machining, shrinking over a mandrel, combinations thereof, or the like.

In certain examples, the proximal ends of the catheters 102 are joined together and form a single-lumen outflow catheter 106, that either extends through a cap 107 at the proximal end of the sheath 104, or is completely located outside of the proximal end of the sheath 104 and connected to a further fluid flow tubing that extends out of the sheath 104. In other examples, the outflow catheter 106 is a separate tube structure that is connected in fluid flow communication with the proximal ends of the catheters 102. The outflow catheter forms a fluid flow path or manifold for connecting the proximal ends of the catheters 102 to the inlet port of a pump 108 (or other suction source), as described below.

The example in FIGS. 1-5 includes a single outflow catheter 106 having a single lumen through and from which all of the catheters 102 extend. That configuration can simplify manufacturing and operation of the system. However, in other examples, more than one outflow catheter 106 may be extended partially through the sheath 104 to contain or guide the plurality of catheters 102. Also, in further examples, the one or more outflow catheters 106 may have more than one lumen.

The one or more outflow catheters 106 may include tubes or tubing that has (or in combination has) an outer diameter or outer dimension (OD) sufficiently small to be received within the ID of the sheath 104. The outflow catheter(s) 106 is extended at least partially through the outer sheath 104, and has at least one lumen and distal end 106a through which the plurality of catheters 102 extend. The distal end sections, including the end length portions 102b of the plurality of catheters 102 extend in the axial direction, out from the distal end(s) 106a of the outflow catheter(s) 106. The outflow catheter(s) 106 may be made of any suitable material and by any suitable manufacturing process, including but not limited to those described with regard to the catheters 102 or the sheath 104. In yet other examples, the outflow catheter(s) 106 may be omitted, and the plurality of catheters 102 are extended through the sheath 104 and to the inlet port 108a of the pump 108.

In particular examples, the system 100 may include or operate with a suction source such as, but not limited to a variable fluid pump device 108, as shown in FIGS. 4 and 5. The pump device 108 has an inlet port side 108a that connects to the proximal ends of the catheters 102, for example, through the outflow catheter 106 (or other tube or manifold connected to the inlet port side 108a of the pump device 108). The pump device 108 has an outlet port 108b that connects in fluid flow communication with a proximal end of the return tube 105.

In certain examples, the pump device 108 is variable and controllable regarding one or more of pump force, pump rate and pump pressure. The pump device 108 may include or operate with control electronics, processing electronics, or both, configured to control the operation of the pump device 108 as described herein. Processing electronics may include one or more electronic processors, microprocessors, CPUs and have associated electronic memory, including non-transient electronic memory devices that store software and/or data for controlling the processing electronics to operate as described herein.

In some examples, the pump device 108 is controlled to operate in a continuous manner at a uniform, constant pumping suction rate. In other examples, the pump 108 is controlled to operate in a periodic or a pulsatile manner, or to operate in pumping bursts, each separated from the next burst by a period of no pumping. For example, the pump device 108 may be controlled to providing pumping action to aspirate for a selected or defined period of time (such as, but not limited to 5 seconds, 10 seconds, 15 seconds, or the like) and then stop pumping for a second selected or defined period of time (such as, but not limited to 10 seconds, 20 seconds, 30 seconds, or the like). In some examples, the pump device 108 is controlled to provide a continuous pumping action for a first period of time (such as, but not limited to 5 minutes, 10 minutes, 15 minutes, or the like), and then cease pumping action (or cease pumping action for a similar duration).

In some examples, one or both of the variable control of the pump device 108 and the storage container(s) or containment devices(s) may be configured to control a rate or timing (or both) of the return of aspirate to the patient to be different from the rate or timing (or both) of aspirate removal from the patient. That configuration can help provide or maintain a reduced renal pressure state at the interface of the renal veins and the inferior vena cava, and to avoid returning the patient to a new steady state.

In some examples, the system 100 also includes one or more storage containers or containment devices connected in fluid flow communication with the return tube 105 or with another portion of the fluid flow path in the system 100. The one or more storage containers or containment devices may be configured to hold up to a particular volume of aspirated blood to be re-introduced into the blood stream of the patient. In certain examples, the storage container or containment device may be included within the pump 108. Alternatively or in addition, one or more storage containers or containment devices may be provided in the fluid flow path, upstream of the inlet port 108a of the pump device 108, or downstream of the outlet port 108b of the pump device 108. In some examples, the one or more storage containers or containment devices may include an agitating device that gently agitates, swirls or circulates blood or other aspirate within the one or more storage containers or containment devices, to avoid or reduce coagulation of the aspirate. In certain examples, the agitating device may include a rotating arm or other rotating structure that imparts a rotational motion of fluid within the one or more storage containers or containment devices.

In particular example, the pump device 108 is configured to be held external or outside of the patient, while being connected to and operating with the catheters 102 implanted within the patient's inferior vena cava, as described herein. In those examples, the outflow catheter 106, the sheath 104 or a length of the catheters 102 may extend through the patient's skin, to connect the external pump to the implanted catheters 102. However, in other examples, the pump 108 may be an implantable pump device that is configured to be implanted within the patient's body, and connected to the catheters 102, internally.

The pump device 108 is configured to apply a suction force through the catheters 102, to provide an aspiration suction at the end length portions 102b of the catheters 102, and to provide a return flow to the return tube 105. In certain examples, a valve or stopcock, such as a 3-way stopcock 112 is provided in the return tube 105 at the return port 104b of the outer sheath 104. The valve or stopcock 112 may be configured to allow a user to remove trapped air bubbles (de-air) the system, or to flush the system with (or input) a heparin-containing saline or other anti-clotting medication. In certain examples, the valve or stopcock 112 may be configured to provide a port or outlet for phlebotomy or blood or serum testing, or an inlet for injecting medication or other substances into the blood stream of the patient. In certain examples, the valve or stopcock 112 may include a safety valve of blood return to the patient.

The pump device 108 may be any fluid pump device suitable for medical or biological fluid. In particular examples, the pump device 108 includes or operates with a switch, sensor or other variable control device S that allows a medical technician or a sensor to accurately adjust the suction pressure to various selectable suction force levels, to adjust the aspiration suction force applied by the catheters 102. In some examples, the device S is connected to the pump 108 by a wired electronic connection. In other examples, the device S is connected to the pump 108 by a wireless, electronic communication connection link such as, but not limited to radio frequency RF, Bluetooth, WiFi, magnetic or induction coupling, or the like. In some examples, the pump device 108 may be a peristaltic pump, a piston pump, a propeller pump, or other pump mechanism suitable for conveying blood from and to a patient.

In certain examples, an electronic monitoring system may be employed to monitor one or more of the patient's biological parameters (such as, but not limited to RBF or GFR), and to automatically adjust the pump pressure (such as to increase or decrease pressure, based on a detected decrease or increase, respectively of the RBF or the GFR). For example, the device S may represent a sensor and processor circuit that detects or monitors the biological parameter and controls the pump 108 to adjust the pumping pressure based on the sensed parameter.

In particular examples, the system 100 is configured as a renal vein aspiration system and may be employed in a process for renal vein aspiration. In those examples, the sheath 104, with the outflow tube 106 and the catheters 102 contained therein, are arranged in the first state (the state shown in FIG. 1), implanted in a patient's inferior vena cava 200, and placed (as shown in FIG. 4) at a position at which the distal end 104a of the sheath 104 (and of the catheters 102) is located adjacent or at the level of the renal veins 202 and 204. In addition, the proximal ends of the catheters 102 are connected to the pump 108 (e.g., directly or through the outflow catheter 106).

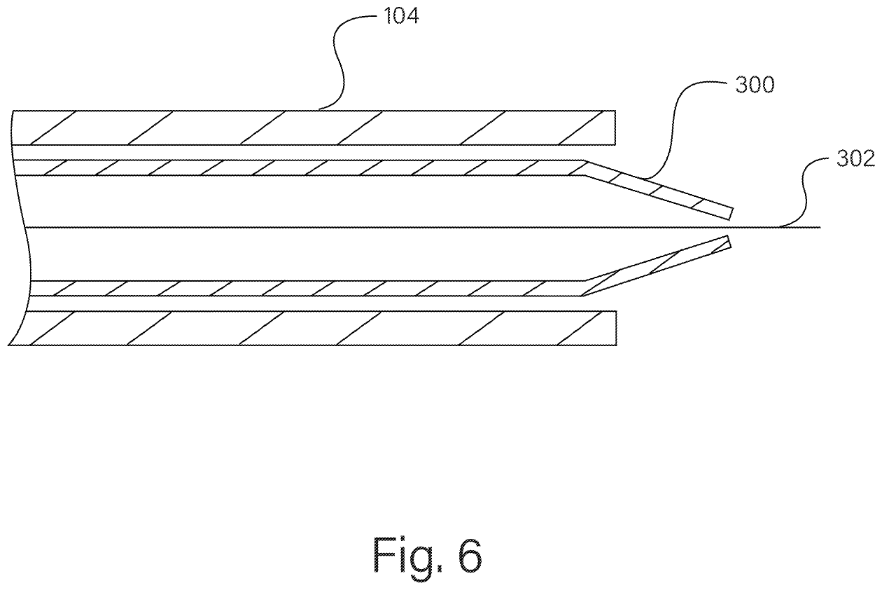

The sheath 104 and catheters 102 may be implanted in a patient's inferior vena cava or iliac vein through any suitable implantation process including, but not limited to endovasular processes. The sheath 104 may be initially fitted with an inner dilator 300 that has a tapered tip and an inner lumen for accommodating a guide wire 302, as shown in FIG. 6. The guide wire may be introduced into the patient, through any suitable venous pathway, such as, but not limited to, through one of the femoral, subclavian or jugular veins and into the inferior vena cave.

The sheath 104, with the inner dilator 300 is advanced over the guidewire to a level of the renal veins. The distal end of the sheath 104, the inner dilator 300 or the end of the guide wire 302 may include a radio-opaque or other detectable feature that can be detected by x-ray, fluoroscope, endoscope or the like during implantation, to adjust the position of the distal end of the sheath 104 relative to the renal veins. Once the distal end of the sheath 104 is adjacent the renal veins, the inner dilator 300 and the guide wire 302 are removed.

Then the catheters 102 and the outflow catheter 106 are introduced into the proximal end of the outer sheath 104 and slid through the lumen of the sheath 104 until the distal ends of the catheters 102 reach at or about the distal end 104a of the sheath 104. At that position, the catheters 102 and the outer sheath 104 are in the first state (the state shown in FIGS. 1 and 4). In some examples, a wrapping material or an introducer tool may hold the catheters 102 together (e.g., in a bundle), to facilitate introducing the catheters 102 into the sheath 104. In a particular example, the plurality of catheters 102 may be held together with a peel-away material that surrounds and forms an introducer around the catheters 102. In those examples, the peel-away material may be configured to be manually peeled off of the catheters 102, as or after the catheters 102 are inserted into the proximal end of the sheath 104. In such examples, the wrapping material or introducer tool helps to hold the catheters 102 together and maintain sufficient rigidity, to facilitate inserting the catheters 102 into the proximal end of the sheath 104 and/or pushing the catheters 102 through the sheath 104.

From that first state, the sheath 104 may be withdrawn from the distal ends 102a of the catheters 102, to the second state (the state shown in FIGS. 2 and 4). As a result, the end length portions 102b of the catheters spring or flare outward, to contact and abut against the wall of the inferior vena cava or the renal veins, adjacent or partially within the interface of the renal veins and the inferior vena cava. In particular, the end length portions 102b of the catheters 102 are located in suitable positions on or adjacent the renal vein ostia, to aspirate fluid from the renal veins. In some examples, the end length portions 102b of the catheters 102 are located to engage the ostia of the renal veins, when sprung or flared outward in a selective fashion, with the catheters 102 in proximal segments of the renal veins.

In that state, the pump 108 may be operated and adjusted via the variable control device S to provide a suitable aspiration suction pressure at the end length portions 102b of the catheters 102, to aspirate blood from the renal veins 202 and 204. The pump 108 may be adjusted to provide aspiration pressure and rate to aspirate a sufficient amount of blood from the renal veins to reduce renal vein pressure and provide conditions that increase renal blood flow (RBF) or renal forward flow in the patient.

The outflow port 108b of the pump 108 is connected to the port 104b of the sheath 104, through the return tube 105, to return the blood to the inferior vena cava of the patient, such as, but not limited to a location proximal or distal to the renal veins, but at a location that does not increase the blood pressure in the renal veins. In other examples, the return may be to the right heart chambers, such as the right atrium, the jugular vein, the iliac vein, the femoral vein, the subclavian vein or another major, central vein. In some example, the return tube 105 is connected to an infusion set or other injection device, for infusing blood back to the patient, into a vein. In any of those or other examples, the return tube 105 may be connected to a dialysis machine for providing a dialysis treatment on the blood, before returning the blood to the patient.

While various exemplary embodiments have been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the function and arrangement of elements without departing from the scope defined by the claims, which includes known equivalents and foreseeable equivalents at the time of filing this patent application.

What is claimed is:

1. An aspiration system comprising:
an outer sheath having an axial dimension, a lumen extending along the axial dimension, and a distal end that is open to the lumen; and
a plurality of catheters located at least partially within the lumen of the outer sheath, each catheter having an axial dimension extending along the axial dimension of the outer sheath, each catheter having a distal end;
wherein the outer sheath and the plurality of catheters are configured to slide relative to each other between a first state in which the distal end of each of the catheters are located within the lumen of the outer sheath, and a second state in which the distal end and an end length section of each of the catheters is extended through the distal end of the outer sheath and is located outside of the lumen of the outer sheath;
wherein the end length section of each of the catheters is configured to spring or flare outward from the axial dimension of the outer sheath relative to the first state, when the outer sheath and the plurality of catheters are in the second state.

2. The aspiration system of claim 1, wherein the end length section of each of the catheters includes a plurality of openings through a catheter wall.

3. The aspiration system of claim 2, wherein the plurality of openings are spaced around and along an axis of each of the catheters.

4. The aspiration system of claim 2, wherein the end length section comprises a length of no more that between about 1 inch and about 3 inches from the distal end of the catheter.

5. The aspiration system of claim 1, further comprising a suction source having an inlet port, wherein the plurality of catheters are connected in fluid flow communication with the inlet port of the suction source for providing an aspiration suction at the distal end or at the end length section of each catheter.

6. The aspiration system of claim 5, wherein the suction source has an outlet port, and wherein the outer sheath includes a return port connected in fluid flow communication with the outlet port of the suction source, for returning an aspirate from the suction source to the lumen of the outer sheath.

7. The aspiration system of claim 5, wherein the suction source comprises a pump having a variably controllable pumping force, rate or pressure.

8. The aspiration system of claim 7, further comprising at least one sensor for sensing a biological condition and providing a signal for controlling the variably controllable pumping force of the pump in response to the biological condition.

9. The aspiration system of claim 5, further comprising at least one storage device for containing an aspirate delivered by the aspiration suction.

10. The aspiration system of claim 1, wherein the outer sheath includes a return port in fluid flow communication with the lumen of the outer sheath, for returning an aspirate to the lumen of the outer sheath.

11. The aspiration system of claim 10, further comprising a return line in fluid flow communication with the return port of the outer sheath, for returning the aspirate to the return port of the outer sheath, and at least one valve or stopcock in the return line for controlling fluid flow through the return line.

12. The aspiration system of claim 1, wherein the end length section of one or more of the catheters has a pre-shaped configuration including at least one curl, at least one bend, a helical configuration, a spiral configuration, or a combination thereof.

13. An aspiration method comprising:
arranging a plurality of catheters at least partially within a lumen of an outer sheath, each catheter having an axial dimension extending along an axial dimension of the outer sheath, each catheter having a distal end located within the lumen of the outer sheath when the plurality of catheters and the outer sheath are in a first state;
placing the distal end of the outer sheath in an inferior vena cava adjacent at least one renal vein, or in an iliac vein, with the outer sheath and the plurality of catheters in the first state;
sliding the outer sheath and the plurality of catheters relative to each other from the first state to a second state in which the distal end and an end length section of each of the catheters is extended through a distal end of the outer sheath and is located outside of the lumen of the outer sheath;
wherein the end length section of each of the catheters is configured to spring or flare outward from the axial dimension of the outer sheath relative to the first state, when the outer sheath and the plurality of catheters are in the second state.

14. The method of claim 13, further comprising abutting the end length section of each of the catheters against a venous wall upon the end length section of each of the catheters being sprung or flared outward in the second state.

15. The method claim 14, further comprising providing a plurality of openings on the end length section of each of the catheters, the plurality of openings being spaced around and along an axis of the catheter.

16. The method of claim 13, further comprising coupling an inlet port of a suction source in fluid flow communication with the plurality of catheters, and providing an aspiration suction at the distal end or at the end length section of each catheter.

17. The method of claim 16, further comprising providing the outer sheath with a return port in fluid flow communication with the lumen of the outer sheath, and coupling an outlet port of the suction source in fluid flow communication with the return port of the outer sheath, and returning an aspirate from the suction source to the lumen of the outer sheath.

18. The method claim 16, wherein the suction source comprises a pump having a variably controllable pumping force, rate or pressure, and the method further comprises providing at least one sensor for sensing a biological condition and controlling the variably controllable pumping force of the pump in response to the biological condition.

19. The method of claim 13, further comprising providing the outer sheath with a return port in fluid flow communication with the lumen of the outer sheath, and returning an aspirate to the lumen of the outer sheath through the return port.

20. The method of claim 19, further comprising connecting a return line in fluid flow communication with the return port of the outer sheath, for returning the aspirate to the return port of the outer sheath, and connecting at least one valve or stopcock in the return line for controlling fluid flow through the return line.
